(19) United States
(12) Patent Application Publication
(10) Pub. No.: US 2026/0120871 A1
(43) Pub. Date: Apr. 30, 2026

(54) Systems and Methods for Processing Images of Cells to Identify Features of a Nucleus of the Cells

(71) Applicant: IDEXX Laboratories, Inc., Westbrook, ME (US)

(72) Inventors: Christopher Watson, Westbrook, ME (US); Jacob Kesinger, Westbrook, ME (US)

(21) Appl. No.: 19/365,890

(22) Filed: Oct. 22, 2025

Related U.S. Application Data
(60) Provisional application No. 63/712,709, filed on Oct. 28, 2024.

(51) Int. Cl. G16H 50/20; G06T 7/00; G16H 30/40

(52) U.S. Cl. CPC G16H 50/20; G06T 7/0012; G16H 30/40; G06T 2207/10056; G06T 2207/10064; G06T 2207/20081; G06T 2207/20112; G06T 2207/30024

(57) ABSTRACT

An example microscopy system includes a stage for receiving a sample with cells, and an objective lens, a light source, and a detection sensor all in optical communication with the stage. The microscopy system also includes a controller communicatively coupled to the detection sensor and the light source, and the controller includes a processor and a memory with a readable set of instructions, which when executed by the processor, cause the processor to receive an image of the cells, apply a first machine learning algorithm to the image that is arranged to output a corresponding mask image of a nucleus of a cell, apply a second algorithm to the corresponding mask image that is arranged to identify contiguous pixels within the nucleus having a pixel value above a configurable threshold and determine a number of lobes of the nucleus based on a number of groups of the contiguous pixels.

### CROSS-REFERENCE TO RELATED APPLICATION

The present disclosure claims priority to U.S. application number 63/712,709 filed on October 28, 2024, the entire contents of which are herein incorporated by reference.

### FIELD

The present disclosure relates generally to systems and methods for imaging a sample with cells using a microscopy system, and more particularly to, processing the images to identify pixels within the nucleus representative of a number of lobes of the nucleus and/or a number of seams of the nucleus as a representation of nuclear segmentation of the cell.

### BACKGROUND

Automated microscopy techniques produce images of many cells. Image recognition machine-learning methods have been developed to characterize cells and samples. However, outputs of the machine-learning methods are often generic and lack details of underlying rationale. While machine-learning methods can distinguish between types of cells, it is desirable to provide more objective information to support the conclusions derived from machine-learning algorithms.

### SUMMARY

Examples methods and systems herein relate to using machine-learning image based data processing of an image of cells from a sample of a patient to evaluate features of a nucleus of the cells in order to provide interpretable context about the cell image as relates to an output of the machine-learning data processing.

In one example, a microscopy system is described comprising a stage for receiving a sample comprising a plurality of cells, an objective lens in optical communication with the stage, a light source in optical communication with the stage, a detection sensor in optical communication with the stage, and a controller communicatively coupled to the detection sensor and the light source. The controller comprises a processor and a memory comprising a readable set of instructions, which when executed by the processor, cause the processor to receive at least one image of the plurality of cells, and apply a first machine learning algorithm to the at least one image. The first machine learning algorithm comprises an image recognition diagnostic model trained on a first set of medical training data and arranged to output a corresponding mask image of a nucleus of one of the plurality of cells. The readable set of instructions are further executable by the processor to apply a second algorithm to the corresponding mask image, and the second algorithm is arranged to identify contiguous pixels within the nucleus having a pixel value above a configurable threshold, and determine a number of lobes of the nucleus based at least in part on a number of groups of the contiguous pixels having the pixel value above the configurable threshold.

In another example, a computing device is described comprising one or more processors, and non-transitory computer readable medium having stored therein instructions that when executed by the one or more processors, causes the computing device to perform functions comprising receiving at least one image of a plurality of cells from a sample, applying a first machine learning algorithm to the at least one image and the first machine learning algorithm comprising an image recognition diagnostic model trained on a first set of medical training data and arranged to output a corresponding mask image of a nucleus of one of the plurality of cells. The functions also comprise applying a second algorithm to the corresponding mask image, and the second algorithm is arranged to identify contiguous pixels within the nucleus having a pixel value above a configurable threshold, and determine a number of lobes of the nucleus based at least in part on a number of groups of the contiguous pixels having the pixel value above the configurable threshold.

In another example, a non-transitory computer readable medium is described having stored thereon instructions, that when executed by one or more processors of a computing device, cause the computing device to perform functions. The functions comprise receiving at least one image of a plurality of cells from a sample, and applying a first machine learning algorithm to the at least one image. The first machine learning algorithm comprises an image recognition diagnostic model trained on a first set of medical training data and arranged to output a corresponding mask image of a nucleus of one of the plurality of cells. The functions also comprise applying a second algorithm to the corresponding mask image, and the second algorithm is arranged to identify contiguous pixels within the nucleus having a pixel value above a configurable threshold, and determine a number of lobes of the nucleus based at least in part on a number of groups of the contiguous pixels having the pixel value above the configurable threshold.

The features, functions, and advantages that have been discussed can be achieved independently in various examples or may be combined in yet other examples. Further details of the examples can be seen with reference to the following description and drawings.

### BRIEF DESCRIPTION OF THE FIGURES

The novel features believed characteristic of the illustrative examples are set forth in the appended claims. The illustrative examples, however, as well as a preferred mode of use, further objectives and descriptions thereof, will best be understood by reference to the following detailed description of an illustrative example of the present disclosure when read in conjunction with the accompanying drawings, wherein:

FIG. 1 illustrates an example of a system 100, according to an example implementation.

FIG. 2 illustrates an example of the controller 110 in FIG. 1, according to an example implementation.

FIG. 3 conceptually illustrates an image 160 output to the controller 110 on a fluorescence channel, according to an example implementation.

FIG. 4 conceptually illustrates a corresponding mask image 162 of the nucleus of a cell, according to an example implementation.

FIG. 5 conceptually illustrates a binary mask image 164 of the nucleus of a cell, according to an example implementation.

FIG. 6 conceptually illustrates an image 180 output to the controller 110 on a fluorescence channel, according to an example implementation.

FIG. 7 conceptually illustrates a second boundary mask image 182 of the nucleus, according to an example implementation.

FIG. 8 conceptually illustrates a binary mask image 184 of the convex hull of the nucleus, according to an example implementation.

FIG. 9 conceptually illustrates a void mask image 186, according to an example implementation.

FIG. 10 conceptually illustrates a resulting number of contiguous groups or patches of pixels representative of seams of the nucleus of the cell, according to an example implementation.

FIG. 11 shows a flowchart of an example of a method 200 for characterizing cells, according to an example implementation.

### DETAILED DESCRIPTION

Disclosed examples will now be described more fully hereinafter with reference to the accompanying drawings. Indeed, several different examples may be described and should not be construed as limited to the examples set forth herein. Rather, these examples are described so that this disclosure will be thorough and complete and will fully convey the scope of the disclosure to those skilled in the art.

Within examples described herein, technical solutions are provided to assist with generating interpretive context data to support outputs of a machine-learning based classification algorithm used to classify cells in images output from a digital microscopy system. Some image recognition machine-learning methods have been developed to characterize cells and samples. However, outputs of the machine-learning methods are often generic and lack details of underlying rationale. Examples systems and methods described herein are utilized to process received images in a technical manner to output supporting data for the classification output.

In one example, classifying white blood cells relies heavily on a shape of their nucleus. Thus, to improve diagnostic accuracy, the shape of a nucleus of a cell distinguishes between different cell types and cell features. For instance, some nuclei may exhibit a lobular or nodular shape, and an extent of lobulation is a factor in cell classification. Consequently, an image processing algorithm capable of detecting the number and arrangement of nuclear lobes and seams in an image is desirable.

Neutrophils (a specific type of white blood cell) more than other types of white blood cells, have a high number of lobes in the cell nucleus. Additionally, mature neutrophils exhibit more lobes than immature neutrophils. More generally, toxic or immature neutrophils have an appearance noticeably different from mature and healthy neutrophils resulting in a more segmented look. Elevated counts of immature neutrophils, known clinically as “left shift”, can be an indication of an inflammatory response and/or an infection. Thus, measuring a number of lobes in cells contributes to identifying such cells algorithmically, and assists with a diagnostic conclusion as well.

As a result, to assist in classifying neutrophils, there is a need for an algorithm that quantifies a degree of nuclear segmentation in a clear and interpretable manner. Example systems and methods described herein perform image processing of cells to characterize nuclear shape by converting cell images into a format enabling counting of nuclear lobes and seams present in a cell image. A higher number of lobes and seams indicates a higher degree of nuclear segmentation. Lobe detection includes identifying bright “islands” in a fluorescence channel of the nucleus, and seam detection is based on finding large voids in a convex hull of the nucleus, for example.

Example systems include a microscopy system that images samples of a patient and processes the images to classify cells and output diagnostic information. Some classifications of cells are accomplished using deep learning classification models, and additional image processing is then performed to provide interpretable measures of nuclear segmentation, giving pathologists confidence in the classification of neutrophils.

Implementations of this disclosure thus provide technological improvements that are particular to computer technology, for example, those concerning image processing, validation, verification, and providing assurances of classifications made by trained machine-learning algorithms. Computer-specific technological problems, such as executing machine-learning logic in beneficial ways, can be wholly or partially solved by implementations of this disclosure. For example, implementation of this disclosure allows for outputs of a machine-learning cell classification algorithm to be supplemented by associating any one or more numerous interpretative context data about the cell (e.g., number of lobes or seams) as further data indicative of a cell type. When the interpretative context data maps to the same cell type as a classification output from execution of the machine-learning algorithm, validation is complete.

The systems and methods of the present disclosure further address problems particular to computer devices and operation of digital imaging instruments, for example, those concerning analysis of captured images. Utilizing machine-learning algorithms, trained on manually labeled images, enables a more immediate and normalized analysis of the data. Thus, analysis of the diagnostic data can occur in a manner that is efficient and takes into account all patients’ needs. Implementations of this disclosure introduce new and efficient improvements in the ways in which data output from digital imaging instruments is analyzed to validate classification results, for example, in an automated and unbiased manner.

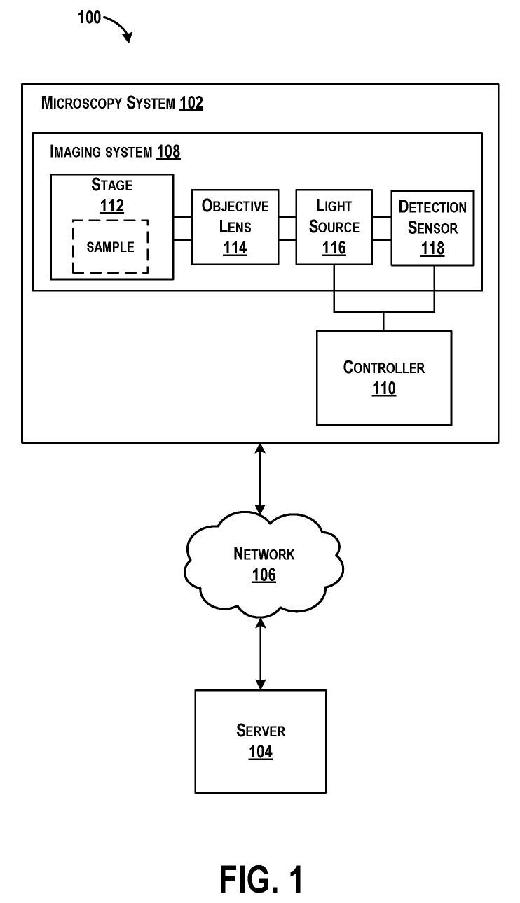

Referring now to the figures, FIG. 1 illustrates an example of a system 100, according to an example implementation. The system 100 includes a microscopy system 102, a server 104, and a network 106. The microscopy system 102 is accessible by the server 104 through the network 106.

In embodiments, the microscopy system 102 includes an imaging system 108 and a controller 110. In other embodiments, the microscopy system 102 includes the imaging system 108 and the controller 110 is a separate component such that the microscopy system 102 is in communication with the controller 110 via a direct wired or wireless communication.

The microscopy system 102 is a form of a diagnostic testing instrument operable to perform diagnostic testing of samples of patients, such as veterinary patients for example. Within examples, the microscopy system 102 includes additional components or is a component of a larger testing instrument. Examples of forms of the microscopy system 102 include any one or combination of veterinary analyzers operable to conduct a diagnostic test of a sample of a patient, and can include without limitation, a clinical chemistry analyzer, a hematology analyzer, a microscopic analyzer, a urine analyzer, an immunoassay reader, a sediment analyzer, a blood analyzer, a digital radiology machine, and/or the like.

In the system 100, the network 106 (e.g., Internet) provides access to the microscopy system 102 and the server 104 for all network-connected components. Communication with the digital microscopy system 102 and the server 104 and/or with the network 106 may be wired or wireless communication (e.g., some components may be in wired Ethernet communication and others may use Wi-Fi communication).

The imaging system 108 of the microscopy system 102 includes a stage 112 for receiving a sample that includes a plurality of cells, an objective lens 114 in optical communication with the stage 112, a light source 116 in optical communication with the stage 112, a detection sensor 118 in optical communication with the stage 112, and a controller 110 communicatively coupled to the detection sensor 118 and the light source 116.

In operation, the microscopy system 102 receives a sample of a patient for processing on the stage 112. In an example, the sample is on a microscope slide for analysis using clinical histopathology based on bright-field microscopy of thinly sliced tissue specimens. In another example, the microscopy system 102 uses slide-free histopathology with direct imaging of intact, minimally processed tissue samples using the imaging system 108, which includes optics and camera components for image processing.

The objective lens 114 both focuses light from the light source 116 onto the stage 112, and directs return light to the detection sensor 118 for image generation. The light source 116 includes a white light source for visibility and imaging, such as one or a number of light-emitting diodes (LEDs). The light source 116 also includes, within examples, filters or other mechanisms to perform near-infrared imaging (NIR), narrow band imaging (NBI), or other fluorescence imaging techniques for excitation of dyes or other labels present in the sample.

The detection sensor 118 can take many forms, and may include an image sensor (e.g., charge-coupled device (CCD) sensor or complementary-metal-oxide-semiconductor (CMOS) sensor) or other camera device adapted to capture return light from the sample to generate an image.

The imaging system 108 captures multiple field of views of the sample into a single image. This process often requires automated image-stitching algorithms that correct field distortion, normalize intensity, align images, and merge them into a single matrix. Image contrast can be distinct in different dynamic ranges, and may be better visualized after high–dynamic-range correction. Color-remapping algorithms can also be implemented to combine and translate novel contrasts into pathologist-familiar color schemes. In examples, the imaging system 108 is capable of using one of many different imaging modalities such as bright-field microscopy, fluorescence imaging, nonlinear microscopy, and structured illumination.

The imaging system 108 outputs one or more images of the sample to the controller 110 for analysis. In some examples, the imaging system 108 additionally or alternatively outputs one or more images of the sample to the server 104 for analysis. In still other examples, the imaging system 108 outputs one or more images of the sample to both the controller 110 and the server 104 for analysis where each of the controller 110 and the server 104 perform portions of the analysis. Any image analysis described herein may be performed by the controller 110 (either internal to the microscopy system 102 or a component separate from the microscopy system 102), by the server 104, or portions may be performed by the controller 110 and the server 104 in communication via the network 106.

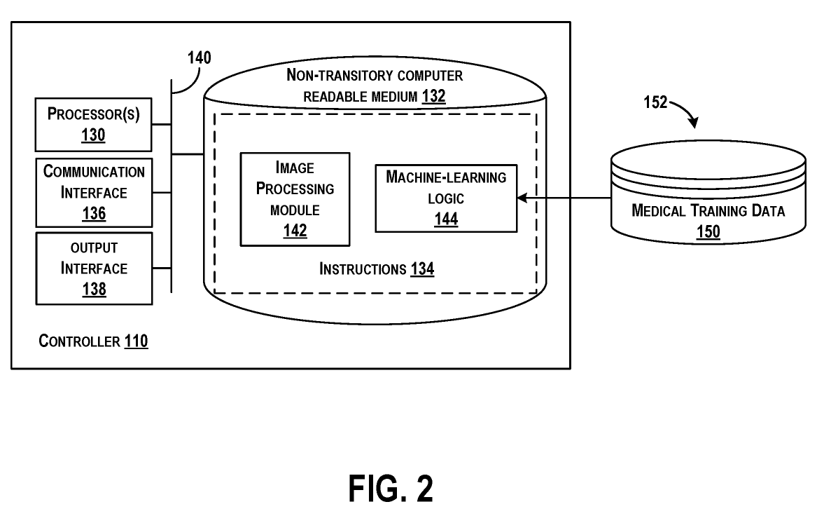

FIG. 2 illustrates an example of the controller 110 in FIG. 1, according to an example implementation. Within examples herein, functions described for processing outputs of the imaging system 108 are performed by the controller 110, by the server 104, or by a combination of the controller 110 and the server 104. Thus, although FIG. 2 illustrates the controller 110, the components of the server 104 are the same as the components of the controller 110 within some examples, depending on where a function is programmed to be performed in a specific implementation.

In addition, within some examples herein, the controller 110 takes the form of a computing device, and the terms controller and computing device are used interchangeably.

The controller 110 includes one or more processor(s) 130, and non-transitory computer readable medium 132 having stored therein instructions 134 that when executed by the one or more processor(s) 130, causes the controller 110 to perform functions for processing an image or multiple images output from the imaging system 108 of the microscopy system 102, as well as management and control of functionality of the microscopy system 102, for example.

To perform these functions, the controller 110 also includes a communication interface 136, an output interface 138, and each component of the controller 110 is connected to a communication bus 140. The controller 110 may also include hardware to enable communication within the server 104 and between the controller 110 and other devices (not shown). The hardware may include transmitters, receivers, and antennas, for example. The controller 110 may further include a display (not shown).

The communication interface 136 may be a wireless interface and/or one or more wireline interfaces that allow for both short-range communication and long-range communication to one or more networks or to one or more remote devices. Such wireless interfaces may provide for communication under one or more wireless communication protocols, Bluetooth, WiFi (e.g., an institute of electrical and electronic engineers (IEEE) 802.11 protocol), Long-Term Evolution (LTE), cellular communications, near-field communication (NFC), and/or other wireless communication protocols. Such wireline interfaces may include an Ethernet interface, a Universal Serial Bus (USB) interface, or similar interface to communicate via a wire, a twisted pair of wires, a coaxial cable, an optical link, a fiber-optic link, or other physical connection to a wireline network. Thus, the communication interface 136 may be configured to receive input data from one or more devices, and may be configured to send output data to other devices.

The non-transitory computer readable medium 132 includes or takes the form of memory, such as one or more computer-readable storage media that can be read or accessed by the one or more processor(s) 130. The non-transitory computer readable medium 132 can include volatile and/or non-volatile storage components, such as optical, magnetic, organic or other memory or disc storage, which can be integrated in whole or in part with the one or more processor(s) 130. In some examples, the non-transitory computer readable medium 132 is implemented using a single physical device (e.g., one optical, magnetic, organic or other memory or disc storage unit), while in other examples, the non-transitory computer readable medium 132 is implemented using two or more physical devices. The non-transitory computer readable medium 132 thus is a computer readable storage, and the instructions 134 are stored thereon. The instructions 134 include computer executable code.

The one or more processor(s) 130 may be general-purpose processors or special purpose processors (e.g., digital signal processors, application specific integrated circuits, etc.). The one or more processor(s) 130 receive inputs from the communication interface 136 (e.g., x-ray images), and process the inputs to generate outputs that are stored in the non-transitory computer readable medium 132. The one or more processor(s) 130 are configured to execute the instructions 134 (e.g., computer-readable program instructions) that are stored in the non-transitory computer readable medium 132 and are executable to provide the functionality of the controller 110 described herein.

The output interface 138 outputs information for transmission, reporting, or storage, and thus, the output interface 138 may be similar to the communication interface 136 and can be a wireless interface (e.g., transmitter) or a wired interface as well.

Within examples, the instructions 134 include specific software for performing functions including an image processing module 142, and machine-learning logic 144.

In one example operation, the processor(s) 130 execute the instructions 134 stored on the non-transitory computer readable medium 132 to cause the controller 110 to perform functions including receive at least one image of the plurality of cells, apply the machine learning logic 144 to the at least one image to output a corresponding mask image of a nucleus of one of the plurality of cells, apply a second algorithm (e.g., image processing module 142) to the corresponding mask image to: identify contiguous pixels within the nucleus having a pixel value above a configurable threshold and determine a number of lobes of the nucleus based at least in part on a number of groups of the contiguous pixels having the pixel value above the configurable threshold.

The machine-learning logic 144 includes an image recognition diagnostic model trained on a first set of medical training data 150. The medical training data 150 includes, for example, digital microscopy images labeled with cell features and is stored in a database 152.

Execution of the machine-learning logic 144 to perform analysis of the microscopy images removes any human bias and generates normalized results for all inputs.

Referring to the machine-learning logic 144, many types of functionality and neural networks can be employed to perform functions of specific machine-learning algorithms to carry out functionality described herein. In one example, the machine-learning logic 144 use statistical models to generate outputs without using explicit instructions, but instead, by relying on patterns and inferences by processing associated training data.

The machine-learning logic 144 can thus operate according to machine-learning tasks as classified into several categories. In supervised learning, the machine-learning logic 144 build a mathematical model from a set of data that contains both the inputs and the desired outputs. The set of data is sample data known as the “training data”, in order to make predictions or decisions without being explicitly programmed to perform the task. For example, the machine-learning logic 144 utilizes the medical training data 150 within comparisons to identify matches in received image data to the labeled cell image data that are within a similarity threshold. When such a match is found, the labeled cell image data is referenced as a label to be applied to the received cell image and the label is further used for defining the data in the image for classification.

In another category referred to as semi-supervised learning, the machine-learning logic 144 develop mathematical models from incomplete training data, where a portion of the sample input does not have labels. A classification algorithm can then be used when the outputs are restricted to a limited set of values.

In another category referred to as unsupervised learning, the machine-learning logic 144 builds a mathematical model from a set of data that contains only inputs and no desired output labels. Unsupervised learning algorithms are used to find structure in related training data, such as grouping or clustering of data points. Unsupervised learning can discover patterns in data, and can group the inputs into categories.

Alternative machine-learning algorithms may be used to learn and classify types of cell images, such as deep learning though neural networks or generative models. Deep machine-learning may use neural networks to analyze prior test results through a collection of interconnected processing nodes. The connections between the nodes may be dynamically weighted. Neural networks learn relationships through repeated exposure to data and adjustment of internal weights. Neural networks may capture nonlinearity and interactions among independent variables without pre specification. Whereas traditional regression analysis requires that nonlinearities and interactions be detected and specified manually, neural networks perform the tasks automatically.

Still other machine-learning algorithms or functions can be implemented to determine and identify content in cell images, such as any number of classifiers that receives input parameters and outputs a classification (e.g., attributes of the image). Support vector machine, Bayesian network, a probabilistic boosting tree, neural network, sparse auto-encoding classifier, convolutional neural network (e.g., for image-based classifiers) or other known or later developed machine-learning algorithms may be used. Any semi-supervised, supervised, or unsupervised learning may be used. Hierarchal, cascade, or other approaches may be also used.

The machine-learning logic 144 may thus be considered an application of rules in combination with learning from prior labeled data to identify appropriate outputs. Analyzing and relying on prior labeled data allows the machine-learning logic 144 to apply patterns of cell data to received cell images for image content identifications, for example.

Thus, the machine-learning logic 144 take the form of one or a combination of any of the herein described machine-learning algorithms, for example.

As mentioned above, the imaging system 108 of the microscopy system 102 outputs one or more images of the sample to the controller 110 for analysis. The analysis includes identification of many different features of cells in the images, in many different ways, as described below.

### Evaluation of cell features for Lobe Detection

In one example, from a diagnostic standpoint, classifying white blood cells relies heavily on a shape of their nucleus. Thus, to improve diagnostic accuracy, the shape of a nucleus of a cell helps distinguish between different cell types. For instance, some nuclei may exhibit a lobular shape, and the extent of lobulation is a factor in cell classification. Consequently, an algorithm capable of detecting the number and arrangement of nuclear lobes is desirable.

The imaging system 108 of the microscopy system 102 captures an image of a patient sample that includes a plurality of cells, and outputs the image to the controller 110.

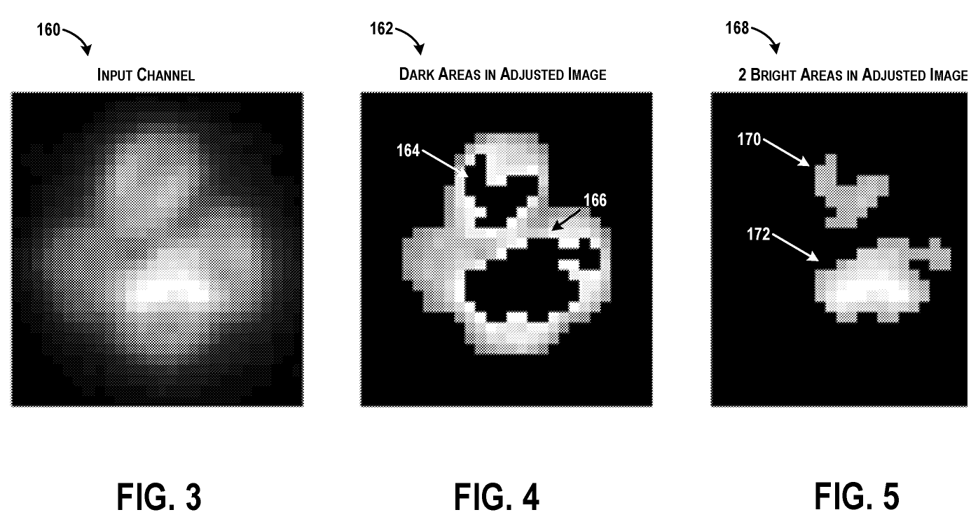

FIG. 3 conceptually illustrates an image 160 output to the controller 110 on a fluorescence channel, according to an example implementation. Initial image processing is performed by the controller 110 to clear up blurriness, rescale the grayscale image, and segment the image to detect the nucleus within the cells by performed image thresholding, such as using Otsu’s method to separate pixels into foreground and background.

Subsequently, the controller 110 applies a first machine learning algorithm to the at least one image, and the first machine learning algorithm comprises an image recognition diagnostic model trained on a first set of medical training data and is arranged to output a corresponding mask image of a nucleus of one of the plurality of cells.

FIG. 4 conceptually illustrates a corresponding mask image 162 of the nucleus of a cell, according to an example implementation. In one example, detection of the cell nucleus is based on ultraviolet fluorescence values in the image. Thus, using training data including labeled images and associated ultraviolet fluorescence values in the image, the first machine learning algorithm outputs the corresponding mask image of the nucleus of a cell in the image. In further examples, the machine-learning logic 144 is further trained using these different factors (labeled with diagnostic outputs) as a basis for recursive training to improve the training model accordingly.

As seen in FIG. 4, an area of the nucleus is given as the corresponding mask image 162 where the nucleus is depicted by dark pixels shown as 164 and 166. From this point, the steps for detecting lobes in an image include considering only pixels within an extent of the nucleus since the algorithm is specifically measuring lobes within the nucleus only.

To assist with the image processing, the controller 110 optionally applies a threshold to pixel values of the corresponding mask image 160 to transform the corresponding mask image 160 into a new binary mask image containing bright areas within the nucleus where separated bright areas are considered as lobe candidates.

FIG. 5 conceptually illustrates a binary mask image 164 of the nucleus of a cell, according to an example implementation. Within examples, the controller 110 converts the corresponding mask image 162 of FIG. 4 into the binary mask image 164 of FIG. 5 by inverting existing dark pixels of FIG. 4 to new bright pixels of FIG. 5 and inverting existing bright pixels of FIG. 4 to new dark pixels of FIG. 5 resulting in the new bright pixels containing the nucleus.

Within examples, the controller 110 applies a second algorithm to the binary mask image 164 that is arranged to identify contiguous pixels within the nucleus having a pixel value above a configurable threshold, and determine a number of lobes of the nucleus based at least in part on a number of groups of the contiguous pixels having the pixel value above the configurable threshold.

In the example in FIG. 5, contiguous pixel groups that have values above a threshold are shown as 170 and 172. A contiguous pixel group includes a grouping of adjacent pixels, each of which has a value above the configurable threshold, and together form a boundary or border with no gaps in the group. Thus, contiguous pixels are touching or directly connected to each other to subsequent touching and adjacent pixels. FIG. 5 illustrates two contiguous pixel groups 170 and 172 and a gap exists between the two groups. These two pixel groups 170 and 172 are candidates for lobes of the nucleus.

The controller 110 counts the number of contiguous groups/patches of pixels, and then measures a size of each of the number of contiguous pixel groups based on a number of pixels contained within each group. The controller 110 executes the second algorithm to remove, from a count of lobe candidates, any groups of the contiguous pixels having the size being below a minimum size. Thus, any groups or patches of pixels deemed to be too small (e.g., by setting a minimum size) are not representative of lobes of a nucleus and are removed from further image processing. Image noise may cause small areas of bright pixels, which would be detected as lobe candidates, but should not be counted as lobes since they are too small and therefore not true lobes.

A resulting count of remaining contiguous groups of pixels is the number of lobes of the nucleus.

Using the image processing techniques described above, a number of lobes of the nucleus are determined in an algorithmic manner, and in a more reliable manner than processing the input image. For example, the corresponding mask image 160 of FIG. 3 generally illustrates a few bright spots and a dark space in between. However, following the image processing for lobe detection, FIG. 5 clearly illustrates two groups of pixels having values above a configurable threshold that are considered representative of lobes of the nucleus of the cell.

### (II) Evaluation of cell features for Seam Detection

In another example, from a diagnostic standpoint, classifying white blood cells relies on a shape of their nucleus and the shape is varied due to presence of seams. Consequently, an algorithm capable of detecting seams of a nucleus is desirable.

The imaging system 108 of the microscopy system 102 captures an image of a patient sample that includes a plurality of cells, and outputs the image to the controller 110.

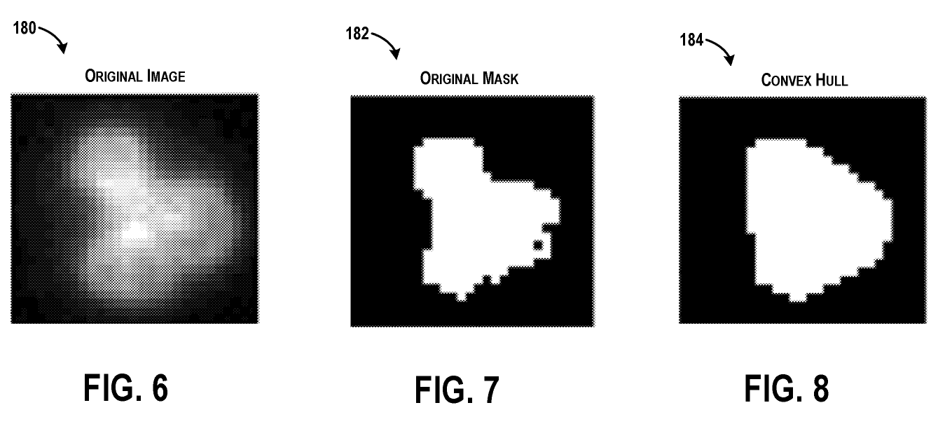

FIG. 6 conceptually illustrates an image 180 output to the controller 110 on a fluorescence channel, according to an example implementation. Similar to FIG. 3 above, initial image processing is performed by the controller 110 to clear up blurriness, rescale the grayscale image, and segment the image to detect the nucleus within the cells by performed image thresholding, such as using Otsu’s method to separate pixels into foreground and background.

Subsequently, the controller 110 applies a first machine learning algorithm to the at least one image, and the first machine learning algorithm comprises an image recognition diagnostic model trained on a first set of medical training data and is arranged to output a second corresponding mask image of a nucleus of one of the plurality of cells that identifies a boundary of the nucleus.

FIG. 7 conceptually illustrates a second boundary mask image 182 of the nucleus, according to an example implementation. The boundary mask image 182 is created by taking a threshold on ultraviolet fluorescence values of the image 180 at a configurable percentage of a maximum pixel value in the image 180. For example, a threshold on the ultraviolet fluorescence values at 55% of the maximum pixel value in the image 180 is performed to identify edges or a boundary of the nucleus, as shown in FIG. 7 as white pixel values.

Following, the controller 110 derives a convex hull of the boundary of the nucleus from the second corresponding mask image 182. FIG. 8 conceptually illustrates a binary mask image 184 of the convex hull of the nucleus, according to an example implementation. The convex hull is a polygon that contains all points or pixels of the nucleus. More specifically, the convex hull is an intersection of all convex sets containing a subset of a Euclidean space including the nucleus.

Next, the controller 110 subtracts the second corresponding mask 182 from the binary mask image 184 (e.g., convex hull), resulting in a void mask image indicating a presence of voids around the nucleus.

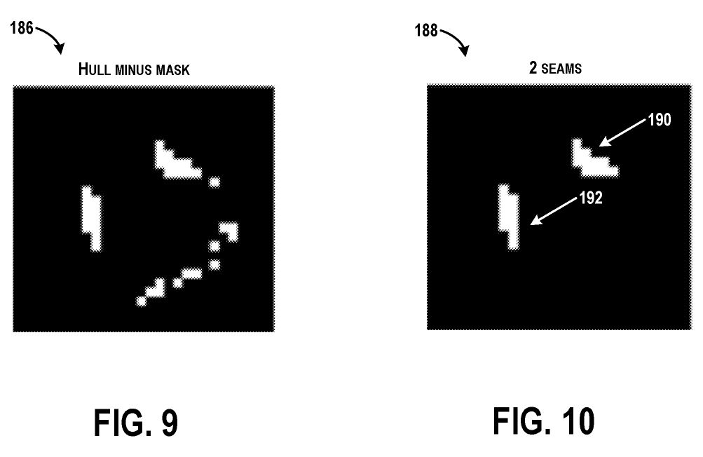

FIG. 9 conceptually illustrates a void mask image 186, according to an example implementation. The white pixels in the void mask image 186 represent voids around the nucleus that are candidate seams.

Following, the controller 110 determines, in the void mask image 186, a second number of groups of second contiguous pixels having a second pixel value above a second configurable threshold, and removes, from the second number of groups, any groups of the second contiguous pixels having a size being below a second minimum size, resulting in a number of seams on the nucleus.

FIG. 10 conceptually illustrates a resulting number of contiguous groups or patches of pixels representative of seams of the nucleus of the cell, according to an example implementation. In FIG. 10, two contiguous groups or patches of pixels 190 and 192 remain that are representative of seams of the nucleus of the cell.

Within examples, the controller 110 utilizes outputs of the lobe detection and seam detection to characterize a degree of nuclear segmentation of the one of the plurality of cells. The controller 110 is then configured to determine a type of the one of the plurality of cells based on the number of lobes of the nucleus. For examples, cells having a given number of lobes are more mature cells. Thus, nuclear segmentation is useful to determine a percentage of neutrophils that are immature, etc.

Further diagnostic procedures and processes are enabled using the image processing methods described herein. For example, the controller 110 processes the at least one image of the plurality of cells to determine the number of lobes of the nucleus for each of the plurality of cells, determines a maturity of each of the plurality of cells based on the number of lobes of the nucleus for each of the plurality of cells, and based on the maturity of the plurality of cells, outputs an indicator of an infection in a patient from which the sample was taken. A number of lobes in the nucleus is helpful in identifying neutrophils. Neutrophils, more than other white blood cells, have a high number of lobes in the cell nucleus. Additionally mature neutrophils exhibit more lobes than immature neutrophils. Elevated counts of immature neutrophils, known clinically as “left shift”, can be an indication of inflammatory response and/or infection. Measuring the number of lobes contributes to identifying such cells algorithmically.

Examples described herein enable an image processing solution, in contrast to a manual subjective evaluation by pathologists, in which machine learning models provide interpretable context about what exactly makes two cells different from each other (e.g., number of lobes and number of seams).

Furthermore, the example methods described herein are applicable to any type of cells, including white blood cells as described in examples that include a nucleus.

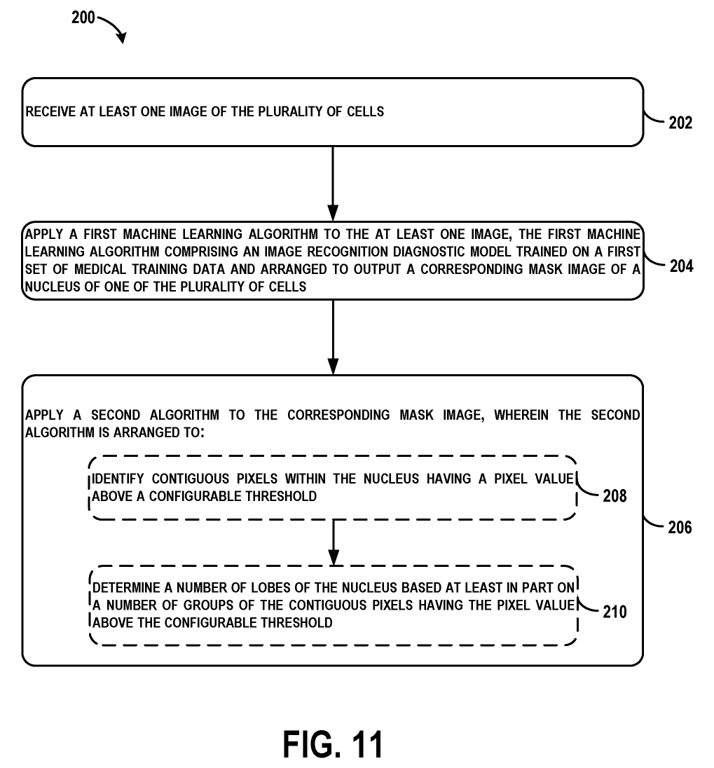

FIG. 11 shows a flowchart of an example of a method 200 for characterizing cells, according to an example implementation. Method 200 shown in FIG. 11 presents an example of a method that could be used with or implemented by the system 100 shown in FIG. 1, the microscopy system 102 in FIG. 1, the controller 110 shown in FIGS. 1-2, or the server 104 shown in FIG. 1, for example. Further, devices or systems may be used or configured to perform logical functions presented in FIG. 11. In some instances, components of the devices and/or systems may be configured to perform the functions such that the components are actually configured and structured (with hardware and/or software) to enable such performance. In other examples, components of the devices and/or systems may be arranged to be adapted to, capable of, or suited for performing the functions, such as when operated in a specific manner. Method 200 may include one or more operations, functions, or actions as illustrated by one or more of blocks 202-210. Although the blocks are illustrated in a sequential order, these blocks may also be performed in parallel, and/or in a different order than those described herein. Also, the various blocks may be combined into fewer blocks, divided into additional blocks, and/or removed based upon the desired implementation.

It should be understood that for this and other processes and methods disclosed herein, flowcharts show functionality and operation of one possible implementation of present examples. In this regard, each block or portions of each block may represent a module, a segment, or a portion of program code, which includes one or more instructions executable by a processor for implementing specific logical functions or steps in the process. The program code may be stored on any type of computer readable medium or data storage, for example, such as a storage device including a disk or hard drive. Further, the program code can be encoded on a computer-readable storage media in a machine-readable format, or on other non-transitory media or articles of manufacture. The computer readable medium may include non-transitory computer readable medium or memory, for example, such as computer-readable media that stores data for short periods of time like register memory, processor cache and Random Access Memory (RAM). The computer readable medium may also include non-transitory media, such as secondary or persistent long term storage, like read only memory (ROM), optical or magnetic disks, compact-disc read only memory (CD-ROM), for example. The computer readable media may also be any other volatile or non-volatile storage systems. The computer readable medium may be considered a tangible computer readable storage medium, for example.

In addition, each block or portions of each block in FIG. 11, and within other processes and methods disclosed herein, may represent computing device(s) and/or circuitry that is wired to or adapted to perform the specific logical functions in the process. Alternative implementations are included within the scope of the examples of the present disclosure in which functions may be executed out of order from that shown or discussed, including substantially concurrent or in reverse order, depending on the functionality involved, as would be understood by those reasonably skilled in the art.

At block 202, the method 200 includes functions to receive at least one image of the plurality of cells. In one example, functions of block 202 include receiving a fluorescence image.

At block 204, the method 200 includes functions to apply a first machine learning algorithm to the at least one image. The first machine learning algorithm comprises an image recognition diagnostic model trained on a first set of medical training data and arranged to output a corresponding mask image of a nucleus of one of the plurality of cells.

At block 206, the method 200 includes functions to apply a second algorithm to the corresponding mask image. At block 208, the second algorithm is arranged to identify contiguous pixels within the nucleus having a pixel value above a configurable threshold. At block 210, the second algorithm is arranged to determine a number of lobes of the nucleus based at least in part on a number of groups of the contiguous pixels having the pixel value above the configurable threshold.

In examples, the second algorithm is further arranged to convert the corresponding mask image into a binary mask image by inverting existing dark pixels to new bright pixels and inverting existing bright pixels to new dark pixels resulting in the new bright pixels containing the nucleus. Subsequently, the second algorithm is arranged to identify, in the binary mask image, the contiguous pixels within the nucleus, among the new bright pixels, having the pixel value above the configurable threshold.

In other examples, the second algorithm is further arranged to determine a size of each of the number of lobes of the nucleus based on a number of pixels contained within each of the groups of the contiguous pixels. Subsequently, the second algorithm is further arranged to remove, from the number of lobes of the nucleus, any groups of the contiguous pixels having the size being below a minimum size.

In some examples, the method 200 additionally includes determining a type of the one of the plurality of cells based on the number of lobes of the nucleus.

In other examples, the method 200 additionally includes processing the at least one image of the plurality of cells to determine the number of lobes of the nucleus for each of the plurality of cells, determining a maturity of each of the plurality of cells based on the number of lobes of the nucleus for each of the plurality of cells, and based on the maturity of the plurality of cells, outputting an indicator of an infection in a patient from which the sample was taken.

In still other examples, the method 200 includes the first machine learning algorithm outputting the corresponding mask image of the nucleus as a first corresponding mask image, and the first machine learning algorithm further outputs a second corresponding mask image of the nucleus of the one of the plurality of cells by identifying a boundary of the nucleus based on taking a threshold on ultraviolet fluorescence values of the at least one image at a configurable percentage of a maximum pixel value in the at least one image. Subsequently, the method 200 includes deriving a convex hull of the boundary of the nucleus from the second corresponding mask image, and subtracting the second corresponding mask from the convex hull, resulting in a void mask image indicating a presence of voids around the nucleus. Further optional functionality includes determining, in the void mask image, a second number of groups of second contiguous pixels having a second pixel value above a second configurable threshold, and removing from the second number of groups, any groups of the second contiguous pixels having a size being below a second minimum size, resulting in a number of seams on the nucleus. Also, still further functionality includes characterizing a degree of nuclear segmentation of the one of the plurality of cells based on the number of lobes of the nucleus and the number of seams on the nucleus.

With reference to FIG. 2, and throughout the disclosure, some components are described as “modules,” “engines”, “models”, or “generators” and such components include or take a form of a general purpose or special purpose hardware (e.g., general or special purpose processors) or firmware configured to execute described functionality, and/or software embodied in a non-transitory computer-readable (storage) medium for execution by one or more processors to perform described functionality.

The description of the different advantageous arrangements has been presented for purposes of illustration and description, and is not intended to be exhaustive or limited to the examples in the form disclosed. Many modifications and variations will be apparent to those of ordinary skill in the art. Further, different advantageous examples may describe different advantages as compared to other advantageous examples. The example or examples selected are chosen and described in order to explain the principles of the examples, the practical application, and to enable others of ordinary skill in the art to understand the disclosure for various examples with various modifications as are suited to the particular use contemplated.

Different examples of the system(s), device(s), and method(s) disclosed herein include a variety of components, features, and functionalities. It should be understood that the various examples of the system(s), device(s), and method(s) disclosed herein may include any of the components, features, and functionalities of any of the other examples of the system(s), device(s), and method(s) disclosed herein in any combination or any sub-combination, and all of such possibilities are intended to be within the scope of the disclosure.

Thus, examples of the present disclosure relate to enumerated clauses (ECs) listed below in any combination or any sub-combination.

EC 1 is a microscopy system comprising: a stage for receiving a sample comprising a plurality of cells; an objective lens in optical communication with the stage; a light source in optical communication with the stage; a detection sensor in optical communication with the stage; and a controller communicatively coupled to the detection sensor and the light source, the controller comprising a processor and a memory comprising a readable set of instructions, which when executed by the processor, cause the processor to: receive at least one image of the plurality of cells; apply a first machine learning algorithm to the at least one image, the first machine learning algorithm comprising an image recognition diagnostic model trained on a first set of medical training data and arranged to output a corresponding mask image of a nucleus of one of the plurality of cells; apply a second algorithm to the corresponding mask image, wherein the second algorithm is arranged to: identify contiguous pixels within the nucleus having a pixel value above a configurable threshold; and determine a number of lobes of the nucleus based at least in part on a number of groups of the contiguous pixels having the pixel value above the configurable threshold.

EC 2 is the microscopy system of EC 1, wherein the readable set of instructions, when executed by the processor, cause the processor to receive the at least one image of the plurality of cells from the detection sensor as a fluorescence image.

EC 3 is the microscopy system of any of ECs 1-2, wherein the second algorithm is further arranged to convert the corresponding mask image into a binary mask image by inverting existing dark pixels to new bright pixels and inverting existing bright pixels to new dark pixels resulting in the new bright pixels containing the nucleus.

EC 4 is the microscopy system of any of ECs 1-3, wherein the second algorithm is arranged to identify, in the binary mask image, the contiguous pixels within the nucleus, among the new bright pixels, having the pixel value above the configurable threshold.

EC 5 is the microscopy system of any of ECs 1-4, wherein the second algorithm is further arranged to determine a size of each of the number of lobes of the nucleus based on a number of pixels contained within each of the groups of the contiguous pixels.

EC 6 is the microscopy system of any of ECs 1-5, wherein the second algorithm is further arranged to remove, from the number of lobes of the nucleus, any groups of the contiguous pixels having the size being below a minimum size.

EC 7 is the microscopy system of any of ECs 1-6, wherein the readable set of instructions, when executed by the processor, further cause the processor to: determine a type of the one of the plurality of cells based on the number of lobes of the nucleus.

EC 8 is the microscopy system of any of ECs 1-7, wherein the readable set of instructions, when executed by the processor, further cause the processor to: process the at least one image of the plurality of cells to determine the number of lobes of the nucleus for each of the plurality of cells; determine a maturity of each of the plurality of cells based on the number of lobes of the nucleus for each of the plurality of cells; and based on the maturity of the plurality of cells, output an indicator of an infection in a patient from which the sample was taken.

EC 9 is the microscopy system of any of ECs 1-8, wherein the first machine learning algorithm outputs the corresponding mask image of the nucleus as a first corresponding mask image, and wherein the first machine learning algorithm further outputs a second corresponding mask image of the nucleus of the one of the plurality of cells by: identifying a boundary of the nucleus based on taking a threshold on ultraviolet fluorescence values of the at least one image at a configurable percentage of a maximum pixel value in the at least one image.

EC 10 is the microscopy system of any of ECs 1-9, wherein the readable set of instructions, when executed by the processor, further cause the processor to: derive a convex hull of the boundary of the nucleus from the second corresponding mask image; and subtract the second corresponding mask from the convex hull, resulting in a void mask image indicating a presence of voids around the nucleus.

EC 11 is the microscopy system of any of ECs 1-10, wherein the readable set of instructions, when executed by the processor, further cause the processor to: determine, in the void mask image, a second number of groups of second contiguous pixels having a second pixel value above a second configurable threshold; and remove, from the second number of groups, any groups of the second contiguous pixels having a size being below a second minimum size, resulting in a number of seams on the nucleus.

EC 12 is the microscopy system of any of ECs 1-11, wherein the readable set of instructions, when executed by the processor, further cause the processor to: characterize a degree of nuclear segmentation of the one of the plurality of cells based on the number of lobes of the nucleus and the number of seams on the nucleus.

EC 13 is a computing device comprising: one or more processors; and non-transitory computer readable medium having stored therein instructions that when executed by the one or more processors, causes the computing device to perform functions comprising: receiving at least one image of a plurality of cells from a sample; applying a first machine learning algorithm to the at least one image, the first machine learning algorithm comprising an image recognition diagnostic model trained on a first set of medical training data and arranged to output a corresponding mask image of a nucleus of one of the plurality of cells; applying a second algorithm to the corresponding mask image, wherein the second algorithm is arranged to: identifying contiguous pixels within the nucleus having a pixel value above a configurable threshold; and determining a number of lobes of the nucleus based at least in part on a number of groups of the contiguous pixels having the pixel value above the configurable threshold.

EC 14 is the computing device of EC 13, wherein the first machine learning algorithm outputs the corresponding mask image of the nucleus as a first corresponding mask image, and wherein the first machine learning algorithm further outputs a second corresponding mask image of the nucleus of the one of the plurality of cells by: identifying a boundary of the nucleus based on taking a threshold on ultraviolet fluorescence values of the at least one image at a configurable percentage of a maximum pixel value in the at least one image.

EC 15 is the computing device of any of ECs 13-14, wherein the functions further comprise: deriving a convex hull of the boundary of the nucleus from the second corresponding mask image; and subtracting the second corresponding mask from the convex hull, resulting in a void mask image indicating a presence of voids around the nucleus.

EC 16 is the computing device of any of ECs 13-15, wherein the functions further comprise: determining, in the void mask image, a second number of groups of second contiguous pixels having a second pixel value above a second configurable threshold; and removing, from the second number of groups, any groups of the second contiguous pixels having a size being below a second minimum size, resulting in a number of seams on the nucleus.

EC 17 is a non-transitory computer readable medium having stored thereon instructions, that when executed by one or more processors of a computing device, cause the computing device to perform functions comprising: receiving at least one image of a plurality of cells from a sample; applying a first machine learning algorithm to the at least one image, the first machine learning algorithm comprising an image recognition diagnostic model trained on a first set of medical training data and arranged to output a corresponding mask image of a nucleus of one of the plurality of cells; applying a second algorithm to the corresponding mask image, wherein the second algorithm is arranged to: identifying contiguous pixels within the nucleus having a pixel value above a configurable threshold; and determining a number of lobes of the nucleus based at least in part on a number of groups of the contiguous pixels having the pixel value above the configurable threshold.

EC 18 is the non-transitory computer readable medium of EC 17, wherein the first machine learning algorithm outputs the corresponding mask image of the nucleus as a first corresponding mask image, and wherein the first machine learning algorithm further outputs a second corresponding mask image of the nucleus of the one of the plurality of cells by: identifying a boundary of the nucleus based on taking a threshold on ultraviolet fluorescence values of the at least one image at a configurable percentage of a maximum pixel value in the at least one image.

EC 19 is the non-transitory computer readable medium of any of ECs 17-18, wherein the functions further comprise: deriving a convex hull of the boundary of the nucleus from the second corresponding mask image; and subtracting the second corresponding mask from the convex hull, resulting in a void mask image indicating a presence of voids around the nucleus.

EC 20 is the non-transitory computer readable medium of any of ECs 17-19, wherein the functions further comprise: determining, in the void mask image, a second number of groups of second contiguous pixels having a second pixel value above a second configurable threshold; and removing, from the second number of groups, any groups of the second contiguous pixels having a size being below a second minimum size, resulting in a number of seams on the nucleus.

By the term “substantially” and “about” used herein, it is meant that the recited characteristic, parameter, or value need not be achieved exactly, but that deviations or variations, including for example, tolerances, measurement error, measurement accuracy limitations and other factors known to skill in the art, may occur in amounts that do not preclude the effect the characteristic was intended to provide. The terms “substantially” and “about” represent the inherent degree of uncertainty that may be attributed to any quantitative comparison, value, measurement, or other representation. The terms “substantially” and “about” are also utilized herein to represent the degree by which a quantitative representation may vary from a stated reference without resulting in a change in the basic function of the subject matter at issue.

It is noted that one or more of the following claims utilize the term “wherein” as a transitional phrase. For the purposes of defining the present invention, it is noted that this term is introduced in the claims as an open-ended transitional phrase that is used to introduce a recitation of a series of characteristics of the structure and should be interpreted in like manner as the more commonly used open-ended preamble term “comprising.”

What is claimed is:

1. A microscopy system comprising:
a stage for receiving a sample comprising a plurality of cells;
an objective lens in optical communication with the stage;
a light source in optical communication with the stage;
a detection sensor in optical communication with the stage; and
a controller communicatively coupled to the detection sensor and the light source, the controller comprising a processor and a memory comprising a readable set of instructions, which when executed by the processor, cause the processor to:
receive at least one image of the plurality of cells;
apply a first machine learning algorithm to the at least one image, the first machine learning algorithm comprising an image recognition diagnostic model trained on a first set of medical training data and arranged to output a corresponding mask image of a nucleus of one of the plurality of cells;
apply a second algorithm to the corresponding mask image, wherein the second algorithm is arranged to:
identify contiguous pixels within the nucleus having a pixel value above a configurable threshold; and
determine a number of lobes of the nucleus based at least in part on a number of groups of the contiguous pixels having the pixel value above the configurable threshold.

2. The microscopy system of claim 1, wherein the readable set of instructions, when executed by the processor, cause the processor to receive the at least one image of the plurality of cells from the detection sensor as a fluorescence image.

3. The microscopy system of claim 1, wherein the second algorithm is further arranged to convert the corresponding mask image into a binary mask image by inverting existing dark pixels to new bright pixels and inverting existing bright pixels to new dark pixels resulting in the new bright pixels containing the nucleus.

4. The microscopy system of claim 3, wherein the second algorithm is arranged to identify, in the binary mask image, the contiguous pixels within the nucleus, among the new bright pixels, having the pixel value above the configurable threshold.

5. The microscopy system of claim 1, wherein the second algorithm is further arranged to determine a size of each of the number of lobes of the nucleus based on a number of pixels contained within each of the groups of the contiguous pixels.

6. The microscopy system of claim 5, wherein the second algorithm is further arranged to remove, from the number of lobes of the nucleus, any groups of the contiguous pixels having the size being below a minimum size.

7. The microscopy system of claim 1, wherein the readable set of instructions, when executed by the processor, further cause the processor to:
determine a type of the one of the plurality of cells based on the number of lobes of the nucleus.

8. The microscopy system of claim 1, wherein the readable set of instructions, when executed by the processor, further cause the processor to:
process the at least one image of the plurality of cells to determine the number of lobes of the nucleus for each of the plurality of cells;
determine a maturity of each of the plurality of cells based on the number of lobes of the nucleus for each of the plurality of cells; and
based on the maturity of the plurality of cells, output an indicator of an infection in a patient from which the sample was taken.

9. The microscopy system of claim 1, wherein the first machine learning algorithm outputs the corresponding mask image of the nucleus as a first corresponding mask image, and wherein the first machine learning algorithm further outputs a second corresponding mask image of the nucleus of the one of the plurality of cells by:
identifying a boundary of the nucleus based on taking a threshold on ultraviolet fluorescence values of the at least one image at a configurable percentage of a maximum pixel value in the at least one image.

10. The microscopy system of claim 9, wherein the readable set of instructions, when executed by the processor, further cause the processor to:
derive a convex hull of the boundary of the nucleus from the second corresponding mask image; and
subtract the second corresponding mask from the convex hull, resulting in a void mask image indicating a presence of voids around the nucleus.

11. The microscopy system of claim 10, wherein the readable set of instructions, when executed by the processor, further cause the processor to:
determine, in the void mask image, a second number of groups of second contiguous pixels having a second pixel value above a second configurable threshold; and
remove, from the second number of groups, any groups of the second contiguous pixels having a size being below a second minimum size, resulting in a number of seams on the nucleus.

12. The microscopy system of claim 11, wherein the readable set of instructions, when executed by the processor, further cause the processor to:
characterize a degree of nuclear segmentation of the one of the plurality of cells based on the number of lobes of the nucleus and the number of seams on the nucleus.

13. A computing device comprising:
one or more processors; and
non-transitory computer readable medium having stored therein instructions that when executed by the one or more processors, causes the computing device to perform functions comprising:
receiving at least one image of a plurality of cells from a sample;
applying a first machine learning algorithm to the at least one image, the first machine learning algorithm comprising an image recognition diagnostic model trained on a first set of medical training data and arranged to output a corresponding mask image of a nucleus of one of the plurality of cells;
applying a second algorithm to the corresponding mask image, wherein the second algorithm is arranged to:
identify contiguous pixels within the nucleus having a pixel value above a configurable threshold; and
determine a number of lobes of the nucleus based at least in part on a number of groups of the contiguous pixels having the pixel value above the configurable threshold.

14. The computing device of claim 13, wherein the first machine learning algorithm outputs the corresponding mask image of the nucleus as a first corresponding mask image, and wherein the first machine learning algorithm further outputs a second corresponding mask image of the nucleus of the one of the plurality of cells by:
identifying a boundary of the nucleus based on taking a threshold on ultraviolet fluorescence values of the at least one image at a configurable percentage of a maximum pixel value in the at least one image.

15. The computing device of claim 14, wherein the functions further comprise:
deriving a convex hull of the boundary of the nucleus from the second corresponding mask image; and
subtracting the second corresponding mask from the convex hull, resulting in a void mask image indicating a presence of voids around the nucleus.

16. The computing device of claim 15, wherein the functions further comprise:
determining, in the void mask image, a second number of groups of second contiguous pixels having a second pixel value above a second configurable threshold; and
removing, from the second number of groups, any groups of the second contiguous pixels having a size being below a second minimum size, resulting in a number of seams on the nucleus.

17. A non-transitory computer readable medium having stored thereon instructions, that when executed by one or more processors of a computing device, cause the computing device to perform functions comprising:
receiving at least one image of a plurality of cells from a sample;
applying a first machine learning algorithm to the at least one image, the first machine learning algorithm comprising an image recognition diagnostic model trained on a first set of medical training data and arranged to output a corresponding mask image of a nucleus of one of the plurality of cells;
applying a second algorithm to the corresponding mask image, wherein the second algorithm is arranged to:
identify contiguous pixels within the nucleus having a pixel value above a configurable threshold; and
determine a number of lobes of the nucleus based at least in part on a number of groups of the contiguous pixels having the pixel value above the configurable threshold.

18. The non-transitory computer readable medium of claim 17, wherein the first machine learning algorithm outputs the corresponding mask image of the nucleus as a first corresponding mask image, and wherein the first machine learning algorithm further outputs a second corresponding mask image of the nucleus of the one of the plurality of cells by:
identifying a boundary of the nucleus based on taking a threshold on ultraviolet fluorescence values of the at least one image at a configurable percentage of a maximum pixel value in the at least one image.

19. The non-transitory computer readable medium of claim 18, wherein the functions further comprise:
deriving a convex hull of the boundary of the nucleus from the second corresponding mask image; and
subtracting the second corresponding mask from the convex hull, resulting in a void mask image indicating a presence of voids around the nucleus.

20. The non-transitory computer readable medium of claim 19, wherein the functions further comprise:
determining, in the void mask image, a second number of groups of second contiguous pixels having a second pixel value above a second configurable threshold; and
removing, from the second number of groups, any groups of the second contiguous pixels having a size being below a second minimum size, resulting in a number of seams on the nucleus.
